(19) United States
(12) Patent Application Publication
(10) Pub. No.: US 2017/0100316 A1
(43) Pub. Date: Apr. 13, 2017

(54) COSMETIC SKIN-LIGHTENING COMPOSITION

(71) Applicant: Henkel AG & Co. KGaA, Duesseldorf (DE)

(72) Inventors: Susanne Dickhof, Viersen (DE); Marianne Waldmann-Laue, Monheim (DE); Soraya Heinen, Koeln (DE); Ira Hartmann, Duesseldorf (DE)

(73) Assignee: Henkel AG & Co. KGaA, Duesseldorf (DE)

(21) Appl. No.: 15/288,114

(22) Filed: Oct. 7, 2016

(30) Foreign Application Priority Data
Oct. 12, 2015 (DE) 10 2015 219 713.1

(51) Int. Cl. A61K 8/37; A61Q 19/02; A61K 8/34; A61Q 17/04; A61K 8/67; A61K 8/365; A61K 8/36; A61K 8/97; A61K 8/06; A61K 8/31

(52) U.S. Cl. CPC A61K 8/375; A61K 8/062; A61Q 19/02; A61K 8/342; A61K 8/31; A61K 8/676; A61K 8/365; A61K 8/347; A61K 8/361; A61K 8/97; A61Q 17/04

(57) ABSTRACT

Cosmetic compositions in the form of an oil-in-water emulsion include
a) at least one monoglycerol fatty acid ester A,
b) at least one first polyglycerol difatty acid ester B,
c) at least one second polyglycerol difatty acid ester C which differs from the first polyglycerol difatty acid ester B,
d) at least one fatty acid ester of the general formula (I)

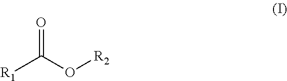

the residues R₁ and R₂ mutually independently denoting a saturated or unsaturated alkyl residue with 16 to 26 carbon atoms, and
e) at least one skin-lightening active substance.

A method includes using such compositions.

### FIELD OF THE INVENTION

The present invention generally relates to cosmetic skin-lightening compositions.

### BACKGROUND OF THE INVENTION

The skin's pigmentation is provided by melanocytes which are to be found in the lowermost layer of the epidermis together with the basal cells as pigment-forming cells which, depending on skin type, occur either in isolation or in accumulations. The melanocytes form melanosomes in which melanin is in turn formed. Increased quantities of melanin are formed by various chemical and/or physical influences, in particular by UV radiation. The melanin is transported via the keratinocytes into the corneocytes (stratum corneum) and gives rise to a brownish to brown-black skin colour. Melanin is formed as the final stage of an oxidative process in which tyrosine is converted with the involvement of the enzyme tyrosinase via a plurality of intermediate stages into brown to brown-black eumelanins (DHICA- and DHI-melanin) or, with the involvement of sulfur-containing compounds, into reddish phaeomelanin. Melanin formation—and thus skin and hair colour—is subject to external influences and, in addition to desired effects (“healthy tan”) can also lead to undesired phenomena. For instance, UV radiation can lead to freckles. Abnormal pigmentation may, however, also occur due to genetic disposition, wound healing or scarring or skin ageing (“age spots”). Hormonally determined disorders may also be responsible for abnormal responses of the melanocytes and for an increased accumulation of melanin in the skin, so resulting in undesired, usually brown, spots on the skin. Since the process of desquamation (superficial detachment of cells or groups of cells from their epithelial association) involves a continual loss of melanin, lightening of the skin can be achieved by inhibiting melanin neosynthesis. Age spots are a sign of skin ageing which demands specific treatment. This is because they involve not only more intense pigmentation of the skin, but also specifically an accumulation of the brownish, waxy pigment lipofuscin (also ageing or wear-and-tear pigment) which arises as the final product from the oxidation of inter alia unsaturated fatty acids of the cell membranes and proteins. The lysosomes are no longer capable of completely breaking down the substance, which accordingly remains as a spot.

Numerous skin-lightening compositions are known from the prior art.

DE 102005031482 A1 proposes skin-lightening agents, inter alia in the form of O/W emulsions, which include ascorbic acid or the physiologically acceptable derivatives or salts thereof together with 8-hexadecene-1,16-dicarboxylic acid, sebacic acid and/or azelaic acid.

Known O/W emulsions for skin lightening, however, often have the disadvantage that their (long-term) storage stability is not always satisfactory.

In particular in the event of temperature fluctuations, it has sometimes been observed that the anti-spot (or whitening) active substances settle out of the formulation and no longer exhibit the desired action to a sufficient extent. It has in the past been possible to counteract this disadvantage by adding relatively large quantities of stabilizers to the formulations. However, a plurality and/or large quantities of different stabilisers in dermatological compositions increase the risk of poorer skin compatibility, for which reason this solution is not always desirable.

It is therefore desirable to provide cosmetic or dermatological oil-in-water emulsions with excellent storage stability. The emulsions should as far as possible be capable of including different whitening active substances (even in relatively high quantities) without suffering any negative impact on the stability thereof (in particular on storage at extremely high or low temperatures and/or with exposure to severe temperature fluctuations). The oil-in-water emulsions should furthermore be very highly skin compatible and exhibit an excellent skin-lightening action. In addition to lightening individual relatively dark pigment spots (or the local accumulations of dark pigment spots), treatment with the oil-in-water emulsions should ideally also result in an overall more uniform skin tone and thus in skin which appears more consistent, more homogeneous and younger.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic composition in the form of an oil-in-water emulsion includes at least one monoglycerol fatty acid ester A, at least one first polyglycerol difatty acid ester B, at least one second polyglycerol difatty acid ester C which differs from the first polyglycerol difatty acid ester B, at least one fatty acid ester of the general formula (I)

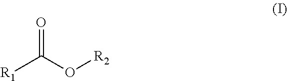

the residues R₁ and R₂ mutually independently denoting a saturated or unsaturated alkyl residue with 16 to 26 carbon atoms, and at least one skin-lightening active substance.

A combination including at least one monoglycerol fatty acid ester A, at least one first polyglycerol difatty acid ester B, at least one second polyglycerol difatty acid ester C which differs from the first polyglycerol difatty acid ester, at least one fatty acid ester of the general formula (I)

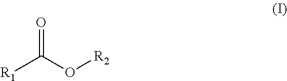

the residues R₁ and R₂ mutually independently denoting a saturated or unsaturated alkyl residue with 16 to 26 carbon atoms, is used for improving the storage stability of a cosmetic composition including at least one skin-lightening active substance; improving the UV-protective action of a cosmetic composition including at least one skin-lightening active substance; improving the absorbability of a cosmetic composition including at least one skin-lightening active substance; and improving the spreadability and reducing the tackiness of a cosmetic composition including at least one skin-lightening active substance.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that cosmetic compositions which, in addition to a skin-lightening active substance, furthermore include a combination of four specific fatty acid esters solve the above-stated problem. Cosmetic compositions on the basis of these combinations are distinguished from conventional prior art compositions by good cosmetic compatibility, good application properties and low tackiness and are furthermore also physically and chemically stable at elevated temperatures over an extended period.

Physical stability here denotes the stability of the rheological properties of the cosmetic composition and the stability thereof against phase separation, including stability against settling of suspended solids.

Chemical stability denotes stability against chemical transformation or decomposition, as may be verified for example by activity testing of the cosmetically active substances d).

The present invention provides:
1. A cosmetic composition in the form of an oil-in-water emulsion including:
   a) at least one monoglycerol fatty acid ester A,
   b) at least one first polyglycerol difatty acid ester B,
   c) at least one second polyglycerol difatty acid ester C which differs from the first polyglycerol difatty acid ester B,
   d) at least one fatty acid ester of the general formula (I)

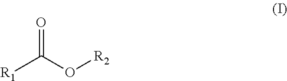

the residues R₁ and R₂ mutually independently denoting a saturated or unsaturated alkyl residue with 16 to 26 carbon atoms,
e) at least one skin-lightening active substance.
2. A cosmetic composition according to point 1, wherein the monoglycerol fatty acid ester A is selected from the group of mono-, di- or triesters of glycerol with behenic acid.
3. A cosmetic composition according to one of the preceding points, wherein the monoglycerol fatty acid ester A is selected from the group of compounds with the INCI name Glyceryl Behenate.
4. A cosmetic composition according to one of the preceding points, wherein the monoglycerol fatty acid ester A is present, relative to the total weight of the compositions, in quantities of 0.01 to 6.0 wt. %, preferably in quantities of 0.1 to 4.0 wt. % and in particular in quantities of 0.5 to 2.0 wt. %.
5. A cosmetic composition according to one of the preceding points, wherein the first polyglycerol difatty acid ester B is selected from the group of di-, tri- or tetraglycerol difatty acid esters, preferably from the group of triglycerol difatty acid esters.
6. A cosmetic composition according to one of the preceding points, wherein the first polyglycerol difatty acid ester B is selected from the group of triglycerol difatty acid esters of saturated or unsaturated fatty acids with 16 to 30 carbon atoms, preferably from the group of triglycerol difatty acid esters of fatty acids from the group palmitic acid, palmitoleic acid, oleic acid, cerotic acid and melissic acid.
7. A cosmetic composition according to one of the preceding points, wherein the first polyglycerol difatty acid ester B is selected from the group of compounds with the INCI name PEG-3 Beeswax.
8. A cosmetic composition according to one of the preceding points, wherein the first polyglycerol difatty acid ester B is present, relative to the total weight of the compositions, in quantities of 0.05 to 3.0 wt. %, preferably in quantities of 0.1 to 2.0 wt. % and in particular in quantities of 0.2 to 1.5 wt. %.
9. A cosmetic composition according to one of the preceding points, wherein the second polyglycerol difatty acid ester C is selected from the group of penta-, hexa- or heptaglycerol difatty acid esters, preferably from the group of hexaglycerol difatty acid esters.
10. A cosmetic composition according to one of the preceding points, wherein the second polyglycerol difatty acid ester C is selected from the group of hexaglycerol difatty acid esters of saturated or unsaturated fatty acids with 16 to 30 carbon atoms, preferably from the group of hexaglycerol difatty acid esters of saturated or unsaturated fatty acids with 16 to 24 carbon atoms and in particular from the group of hexaglycerol difatty acid esters of stearic acid.
11. A cosmetic composition according to one of the preceding points, wherein the second polyglycerol difatty acid ester C is selected from the group of compounds with the INCI name PEG-3 Distearate.
12. A cosmetic composition according to one of the preceding points, wherein the second polyglycerol difatty acid ester C is present, relative to the total weight of the compositions, in quantities of 0.2 to 8.0 wt. %, preferably of 0.4 to 6.0 wt. % and in particular of 0.6 to 4.0 wt. %.
13. A cosmetic composition according to one of the preceding points, wherein in the general formula (I) the residues R₁ and R₂ mutually independently denote an unsaturated alkyl residue with 16 to 26 carbon atoms, preferably an unsaturated alkyl residue with 18 to 24 carbon atoms.
14. A cosmetic composition according to one of the preceding points, wherein the fatty acid ester of the general formula (I) is present, relative to the total weight of the compositions, in quantities of 0.05 to 3.0 wt. %, preferably in quantities of 0.1 to 2.0 wt. % and in particular in quantities of 0.2 to 1.5 wt. %.
15. A cosmetic composition according to one of the preceding points, wherein the skin-lightening active substance e) which is present is ascorbic acid, ascorbic acid salts, ascorbic acid glycosides, ascorbic acid glycoside salts, organic ascorbic acid esters, salts of organic ascorbic acid esters, inorganic ascorbic acid esters, salts of organic ascorbic acid esters, 2-(4-hydroxyphenoxy)propionic acid, salts of 2-(4-hydroxyphenoxy)propionic acid, resorcinol derivatives, saturated or unsaturated, linear C₉₋₁₈ dicarboxylic acid (salts), extracts of Olea europaea or a mixture of these active substances.
16. A cosmetic composition according to one of the preceding points, wherein the skin-lightening active substance e) which is present is ascorbic acid, ascorbic acid salts, ascorbic acid glycosides, ascorbic acid glycoside salts, organic ascorbic acid esters, salts of organic ascorbic acid esters, inorganic ascorbic acid esters and/or salts of organic ascorbic acid esters, in particular ascorbic acid and/or at least one of the compounds known by the INCI names Ascorbyl Tetraisopalmitate, Magnesium Ascorbyl Phosphate and/or Ascorbyl Glucoside.
17. A cosmetic composition according to one of the preceding points, wherein the skin-lightening active substance e) which is present is 2-(4-hydroxyphenoxy)propionic acid and/or an alkali metal or ammonium salt of 2-(4-hydroxyphenoxy)propionic acid, in particular 2-(4-hydroxyphenoxy)propionic acid.
18. A cosmetic composition according to one of the preceding points, wherein the skin-lightening active substance e) which is present is at least one plant extract known by the INCI name Olea europaea Leaf Extract.
19. A cosmetic composition according to one of the preceding points, wherein the skin-lightening active substance e) which is present is a mixture of
   ascorbic acid and Ascorbyl Glucoside,
   at least one plant extract known by the INCI name Olea europaea Leaf Extract, and
   (R)-2-(4-hydroxyphenoxy)propionic acid and/or the alkali metal or ammonium salt thereof
20. A cosmetic composition according to one of the preceding points, wherein the skin-lightening active substance e) which is present is at least one compound known by the INCI name Phenylethyl Resorcinol and/or at least one linear C₉₋₁₈ dicarboxylic acid, preferably 8-hexadecene-1,16-dicarboxylic acid, 7-tetradecene-1,14-dicarboxylic acid, 9-octadecene-1,18-dicarboxylic acid, 6-dodecene-1,12-dicarboxylic acid, 5-decene-1,10-dicarboxylic acid, decanedioic acid (sebacic acid) and/or nonanedioic acid (azelaic acid), particularly preferably Phenylethyl Resorcinol and/or 9-octadecene-1,18-dicarboxylic acid (INCI name: Octadecenedioic Acid).
21. A cosmetic composition according to one of the preceding points, wherein the skin-lightening active substance e) is selected from the group ascorbic acid, 2-(4-hydroxyphenoxy)propionic acid, Phenylethyl Resorcinol, Octadecenedioic Acid, Olea europaea extract, Ascorbyl Tetraisopalmitate, Magnesium Ascorbyl Phosphate and Ascorbyl Glucoside.
22. A cosmetic composition according to one of the preceding points, wherein the skin-lightening active substance e) is present, relative to the total weight of the compositions, in quantities of 0.02 to 9.0 wt. %, preferably 0.03 to 8.0 wt. %, more preferably 0.04 to 7.0 wt. % and in particular 0.05 to 6.0 wt. %.
23. A cosmetic composition according to one of the preceding points, wherein the composition furthermore includes
   f) a linear fatty alcohol, preferably a linear fatty alcohol with 14 to 18 carbon atoms and in particular a linear fatty alcohol with 16 carbon atoms.
24. A cosmetic composition according to point 23, wherein the linear fatty alcohol is present, relative to the total weight of the compositions, in quantities of 0.01 to 5.0 wt. %, preferably in quantities of 0.05 to 3.0 wt. % and in particular in quantities of 0.1 to 1.0 wt. %.
25. A cosmetic composition according to one of the preceding points, wherein the composition furthermore includes
   g) a linear alkane with 11 to 18 carbon atoms, preferably a linear alkane with 11 to 13 carbon atoms and in particular a linear alkane with 11 and/or 13 carbon atoms.
26. A cosmetic composition according to point 25, wherein the linear alkane is present, relative to the total weight of the compositions, in quantities of 0.1 to 10 wt. %, preferably of 0.2 to 7.0 wt. % and in particular of 0.5 to 5.0 wt. %.
27. A cosmetic composition according to one of the preceding points, wherein the composition furthermore includes at least one organic UV filter h).
28. A cosmetic composition according to one of the preceding points, wherein the composition furthermore includes at least one hydrophobically modified (meth)acrylic acid copolymer.
29. A cosmetic composition according to point 28, wherein the hydrophobically modified (meth)acrylic acid copolymer is attributable to
   at least one monomer from the group acrylic acid, methacrylic acid, C₁-C₆ alkyl acrylic acid esters or C₁-C₆ alkyl methacrylic acid esters, and
   at least one monomer from the group of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates, preferably from the group of C₁₀₋₃₀ alkyl acrylates.
30. A cosmetic composition according to one of points 28 or 29, wherein the hydrophobically modified (meth)acrylic acid copolymer is present in quantities of 0.05 to 2.0 wt. %, preferably of 0.1 to 1.2 wt. % and in particular of 0.2 to 0.8 wt. %.
31. A cosmetic composition according to one of the preceding points, wherein the composition furthermore includes at least one copolymer of 2-methyl-2[(1-oxo-2-propenyl)amino]-1-propanesulfonic acid.
32. A cosmetic composition according to point 30, wherein the copolymer of 2-methyl-2[(1-oxo-2-propenyl)amino]-1-propanesulfonic acid is attributable to
   2-methyl-2[(1-oxo-2-propenyl)amino]-1-propanesulfonic acid and
   vinylpyrrolidone.
33. A cosmetic composition according to one of points 31 or 32, wherein the copolymer of 2-methyl-2[(1-oxo-2-propenyl)amino]-1-propanesulfonic acid is present, relative to the total weight of the composition, in quantities of 0.05 to 3.0 wt. %, preferably of 0.1 to 2.0 wt. % and in particular of 0.2 to 1.5 wt. %.
34. Use of a combination including
   a) at least one monoglycerol fatty acid ester A,
   b) at least one first polyglycerol difatty acid ester B,
   c) at least one second polyglycerol difatty acid ester C which differs from the first polyglycerol difatty acid ester,
   d) at least one fatty acid ester of the general formula (I)

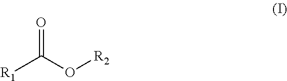

the residues R₁ and R₂ mutually independently denoting a saturated or unsaturated alkyl residue with 16 to 26 carbon atoms,
for
   improving the storage stability of a cosmetic composition including at least one skin-lightening active substance;
   improving the UV-protective action of a cosmetic composition including at least one skin-lightening active substance;
   improving the absorbability of a cosmetic composition including at least one skin-lightening active substance;
   improving the spreadability and reducing the tackiness of a cosmetic composition including at least one skin-lightening active substance.
34. A method for cosmetic skin care, in which a cosmetic composition according to one of points 1 to 32 is applied onto the skin.

The present invention firstly provides a cosmetic composition in the form of an oil-in-water emulsion including:

a) at least one monoglycerol fatty acid ester A,
b) at least one first polyglycerol difatty acid ester B,
c) at least one second polyglycerol difatty acid ester C which differs from the first polyglycerol difatty acid ester,
d) at least one fatty acid ester of the general formula (I)

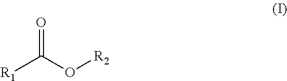

the residues R₁ and R₂ mutually independently denoting a saturated or unsaturated alkyl residue with 16 to 26 carbon atoms,
e) at least one skin-lightening active substance.

The cosmetic compositions according to the invention assume the form of an oil-in-water emulsion and thus include an aqueous fraction. The composition according to the invention is preferably viscous-liquid. Preferred cosmetic compositions are characterised by a water content, relative to the total weight thereof, of 30 to 82 wt. %, preferably 35 to 75 wt. %, in particular 40 to 55 wt. %.

In addition to water, the compositions according to the invention may furthermore include, relative to the total weight thereof, 1 to 15 wt. %, preferably 2 to 12 and in particular 3 to 10 wt. % of at least one alcohol. Preferred alcohols are glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycols, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, polyethylene glycol and mixtures of the above-stated substances. The compositions according to the invention more preferably include glycerol and/or 1,6-hexanediol as the alcohol.

The cosmetic compositions include at least one monoglycerol fatty acid ester A as the first essential component thereof.

Suitable fatty acids are in particular C₁₆₋₂₂ carboxylic acids, preferably saturated, linear C₁₆₋₂₂ carboxylic acids. The monoglycerol fatty acid ester A is more preferentially selected from the group of mono-, di- or triesters of glycerol with behenic acid. One exemplary monoglycerol fatty acid ester A which is particularly advantageous with regard to the action thereof is a mixture of mono-, di- and triglycerides of behenic acid. Mixtures including 10 to 25 wt. % monoglyceryl behenate, 40 to 65 wt. % diglyceryl behenate and 25 to 50 wt. % triglyceryl behenate are particularly preferred.

The monoglycerol fatty acid ester A is more preferentially selected from the group of compounds with the INCI name Glyceryl Behenate. One suitable ester is obtainable under the trade name Compritol® 888 CG (from Gattefosse).

The proportion by weight of the monoglycerol fatty acid ester A in the total weight of cosmetic compositions preferably amounts to from 0.01 to 6.0 wt. %, preferably from 0.1 to 4.0 wt. % and in particular from 0.5 to 2.0 wt. %.

The polyglycerol difatty acid ester B is a further essential component of the cosmetic compositions.

Examples of polyglycerol difatty acid esters B are Polyglyceryl-2 Diisostearate, Polyglyceryl-2 Dioleate, Polyglyceryl-2 Dipolyhydroxystearate, Polyglyceryl-2 Distearate, Polyglyceryl-3 Dicaprate, Polyglyceryl-3 Dicocoate, Polyglyceryl-3 Di-Hydroxystearate, Polyglyceryl-3 Diisostearate, Polyglyceryl-3 Dioleate or Polyglyceryl-3 Distearate.

Preferred polyglycerol difatty acid esters B are selected from the group of di-, tri- or tetraglycerol difatty acid esters, preferably from the group of triglycerol difatty acid esters.

Thanks to the cosmetic and technical action thereof, preferred polyglycerol difatty acid esters B are those from the group of triglycerol difatty acid esters of saturated or unsaturated fatty acids with 16 to 30 carbon atoms, preferably from the group of triglycerol difatty acid esters of fatty acids from the group palmitic acid, palmitoleic acid, oleic acid, cerotic acid and melissic acid.

Examples of polyglycerol difatty acid esters B are Polyglyceryl-2 Dipalmitate, Polyglyceryl-2 Dipalmitoleate, Polyglyceryl-2 Dioleate, Polyglyceryl-2 Dicerotinate, Polyglyceryl-2 Dimelissinate, Polyglyceryl-3 Dipalmitate, Polyglyceryl-3 Dipalmitoleate, Polyglyceryl-3 Dioleate, Polyglyceryl-3 Dicerotinate, Polyglyceryl-3 Dimelissinate, Polyglyceryl-4 Dipalmitate, Polyglyceryl-4 Dipalmitoleate, Polyglyceryl-4 Dioleate, Polyglyceryl-4 Dicerotinate and Polyglyceryl-4 Dimelissinate. Polyglyceryl-3 Dipalmitate, Polyglyceryl-3 Dicerotinate and Polyglyceryl-3 Dimelissinate are more preferred.

Palmitic acid, palmitoleic acid, oleic acid, cerotic acid and melissic acid are components of beeswax and the above-stated polyglycerol difatty acid esters can be produced inter alia by reacting polyglycerol with the free acids of beeswax. More preferred polyglycerol difatty acid esters B are selected from the group of compounds with the INCI name PEG-3 Beeswax.

The proportion by weight of the polyglycerol difatty acid ester B in the total weight of the cosmetic compositions is preferably from 0.05 to 3.0 wt. %, preferably from 0.1 to 2.0 wt. % and in particular from 0.2 to 1.5 wt. %.

The cosmetic compositions include a second polyglycerol difatty acid ester C which differs from the first polyglycerol difatty acid ester B as the third essential component thereof.

Examples of polyglycerol difatty acid esters C are Polyglyceryl-4 Dilaurate, Polyglyceryl-5 Dilaurate, Polyglyceryl-5 Dioleate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-10 Didecanoate, Polyglyceryl-10 Diisostearate, Polyglyceryl-10 Dilaurate, Polyglyceryl-10 Dimyristate, Polyglyceryl-10 Dioleate, Polyglyceryl-10 Dipalmitate and Polyglyceryl-10 Distearate.

Thanks to the advantageous cosmetic and technical action, the second polyglycerol difatty acid ester C is preferably selected from the group of penta-, hexa- or heptaglycerol difatty acid esters, preferably from the group of hexaglycerol difatty acid esters.

Thanks to the cosmetic and technical action thereof, preferred polyglycerol difatty acid esters C are those from the group of hexaglycerol difatty acid esters of saturated or unsaturated fatty acids with 16 to 30 carbon atoms, preferably from the group of hexaglycerol difatty acid esters of saturated or unsaturated fatty acids with 16 to 24 carbon atoms and in particular from the group of hexaglycerol difatty acid esters of stearic acid.

More preferred polyglycerol difatty acid esters C are selected from the group of compounds with the INCI name PEG-6 Distearate.

The proportion by weight of the polyglycerol difatty acid ester C in the total weight of the compositions preferably amounts to from 0.2 to 8.0 wt. %, preferably from 0.4 to 6.0 wt. % and in particular from 0.6 to 4.0 wt. %.

The fatty acid ester of the general formula (I) is a further essential component of the cosmetic compositions. Thanks to the cosmetic and technical advantages thereof, preferred fatty acid esters are those in which the residues R₁ and R₂ in the general formula (I) mutually independently denote an unsaturated alkyl residue with 16 to 26 carbon atoms, preferably an unsaturated alkyl residue with 18 to 24 carbon atoms.

More preferred fatty acid esters of the general formula (I) are from the group of compounds with the INCI name Jojoba Esters.

Preferably, the proportion by weight of the fatty acid ester of the general formula (I) in the total weight of the compositions amounts to 0.05 to 3.0 wt. %, preferably from 0.1 to 2.0 wt. % and in particular from 0.2 to 1.5 wt. %.

Thanks to the advantageous cosmetic action and the high chemical as well as physical storage stability, it is more preferred to use mixtures of polyglycerol difatty acid esters B, polyglycerol difatty acid esters C and fatty acid esters of the general formula (I) which include, relative to the total weight of the mixture
5.0 to 10 wt. % polyglycerol difatty acid esters B,
50 to 75 wt. % polyglycerol difatty acid esters C and
10 to 25 wt. % fatty acid ester of the general formula (I).

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

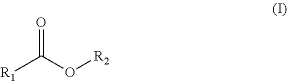

the residues R₁ and R₂ mutually independently denoting an unsaturated alkyl residue with 16 to 26 carbon atoms, and
e) at least one skin-lightening active substance.

In a preferred embodiment, the cosmetic compositions furthermore include as optional component a linear fatty alcohol, preferably a linear fatty alcohol with 14 to 18 carbon atoms, in particular a linear fatty alcohol with 16 carbon atoms.

Relative to the total weight of the compositions, the proportion by weight of the linear fatty alcohol preferably amounts to 0.01 to 5.0 wt. %, preferably from 0.05 to 3.0 wt. % and in particular from 0.1 to 1.0 wt. %.

The compositions according to the invention include at least one skin-lightening active substance as fourth essential component. Said active substance is required for reducing and/or eliminating dark pigment spots from the skin. Preferred quantities of the skin-lightening active substance e) are 0.02 to 9.0 wt. %, preferably 0.03 to 8.0 wt. %, more preferably 0.04 to 7.0 wt. % and in particular 0.05 to 6.0 wt. %.

It has been found that certain skin-lightening active substances are particularly suitable according to the invention because not only can they be excellently combined with components a) to d) and give rise to particularly storage-stable compositions, but in addition to lightening individual, localised dark patches of skin, equalise skin colour over a large area. As a result, treated skin appears fresher and younger.

Particularly suitable skin-lightening active substances are selected from: ascorbic acid, ascorbic acid salts, ascorbic acid glycosides, ascorbic acid glycoside salts, organic ascorbic acid esters, salts of organic ascorbic acid esters, inorganic ascorbic acid esters, salts of organic ascorbic acid esters, 2-(4-hydroxyphenoxy)propionic acid, salts of 2-(4-hydroxyphenoxy)propionic acid, resorcinol derivatives, saturated or unsaturated, linear C₉₋₁₈ dicarboxylic acid (salts), extracts of Olea europaea and mixtures of these active substances. In one preferred embodiment, compositions according to the invention accordingly include at least one active substance from the above-stated group as at least one skin-lightening active substance e).

Suitable ascorbic acid salts should preferably be taken to be alkali metal, ammonium and alkaline earth metal salts. Particularly preferred salts of ascorbic acid which may be used according to the invention as skin-lightening active substance are sodium hydrogen ascorbate, sodium ascorbate, potassium hydrogen ascorbate, potassium ascorbate, ammonium ascorbate, ammonium hydrogen ascorbate, calcium ascorbate, calcium hydrogen ascorbate, magnesium ascorbate and magnesium hydrogen ascorbate.

Suitable ascorbic acid esters for the purposes of the present invention may preferably be selected from esters of ascorbic acid with organic acids according to formula (I) below

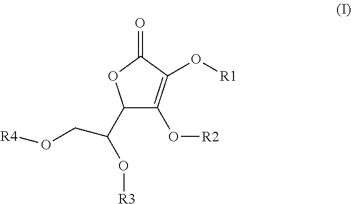

in which one to four of the residues R₁ to R₄ denote the grouping —C(O)—R and the other residues optionally denote hydrogen; and R denotes a straight-chain or branched, saturated or unsaturated alk(en)yl residue with 8 to 24, preferably with 10 to 20 and in particular with 13 to 17 carbon atoms.

The following compounds are particularly preferred esters of ascorbic acid with organic acids: ascorbyl palmitate, ascorbyl isopalmitate, ascorbyl tetraisopalmitate, ascorbyl stearate, ascorbyl isostearate, ascorbyl oleate and/or ascorbyl linoleate. Thanks to its excellent skin penetration capacity, ascorbyl tetraisopalmitate is a more preferred organic ascorbic acid ester.

Further ascorbic acid esters which are suitable for the purposes of the present invention are esters of ascorbic acid with inorganic acids, preferably with phosphoric acid or sulfuric acid. More preferred according to the invention are ascorbyl phosphate and/or ascorbyl sulfate or the alkali or alkaline earth metal salts of ascorbyl phosphate and/or ascorbyl sulfate, such as for example the compounds known by the INCI names Sodium Ascorbyl Phosphate, Magnesium Ascorbyl Phosphate, Sodium Ascorbyl Sulfate and/or Magnesium Ascorbyl Sulfate. Thanks to its excellent stability in aqueous media, Magnesium Ascorbyl Phosphate is a more preferred inorganic ascorbic acid ester.

Ascorbic acid derivatives with glycosidically bound sugars are also more preferentially usable according to the invention. Ascorbyl glucoside and/or one of the physiologically acceptable salts thereof has in particular proved effective in this connection.

More preferred compositions are those which, in order to boost the stability and efficacy of the compositions, include combinations of fat- and water-soluble ascorbic acid (derivatives). In particular, combinations of ascorbic acid with the above-stated ascorbyl phosphate salts and/or ascorbyl glucoside and/or ascorbyl tetraisopalmitate are preferred.

A further preferred embodiment of the invention is accordingly characterised in that the cosmetic compositions include ascorbic acid, ascorbic acid salts, ascorbic acid glycosides, ascorbic acid glycoside salts, organic ascorbic acid esters, salts of organic ascorbic acid esters, inorganic ascorbic acid esters and/or salts of organic ascorbic acid esters as the skin-lightening active substance e). Compositions which are preferred within this embodiment are in particular those which include ascorbic acid and/or at least one of the compounds known by the INCI names Ascorbyl Tetraisopalmitate, Magnesium Ascorbyl Phosphate and/or Ascorbyl Glucoside.

Ascorbic acid and/or the above-stated preferred derivatives and/or salts thereof may be used in the cosmetic compositions according to the invention preferably in quantities of 0.01 to 5.0 wt. %, more preferably of 0.02 to 4.50 wt. %, more preferably of 0.03 to 4.0 wt. %, particularly preferably of 0.04 to 3.50 wt. % and in particular of 0.05 to 3.0 wt. %, wherein the indications of quantity relate to the total weight of the compositions.

More preferred compositions are those which include ascorbic acid and/or at least one of the compounds known by the INCI names Ascorbyl Tetraisopalmitate, Magnesium Ascorbyl Phosphate and/or Ascorbyl Glucoside in the above-stated quantities. In particular, mixtures of ascorbic acid and Ascorbyl Glucoside are preferred.

A further preferred skin-lightening active substance e), which can be excellently combined with the further components a) to d), is an extract of Olea europaea, in particular an extract of the leaves of Olea europaea, as is for example commercially obtainable from various suppliers under the INCI name Olea europaea Leaf Extract.

The extract is produced by methods which are known per se from the prior art preferably from the leaves of the olive tree with a mixture of water and alcohol and subsequent hydrolysis of the extract.

Olive leaves include per gram dry weight on average 60-90 mg of the polyphenol oleuropein (in addition to phenols, flavonoids, organic acids and hydrocarbons) which exhibits very high antioxidative efficacy. Furthermore, however, oleuropein also brings about a significant reduction in the cellular breakdown products lipofuscin which are responsible for the formation of age spots.

In a further preferred embodiment, cosmetic compositions are characterised in that they include at least one plant extract known by the INCI name Olea europaea Leaf Extract as the skin-lightening active substance e).

The extract of Olea europaea, in particular an extract known by the INCI name Olea europaea Leaf Extract may be used in the cosmetic compositions preferably in a quantity of 0.01 to 3.0 wt. %, more preferably of 0.02 to 2.50 wt. %, more preferably of 0.03 to 2.0 wt. %, particularly preferably of 0.04 to 1.50 wt. % and in particular of 0.05 to 1.0 wt. %, wherein the indications of quantity relate to the total weight of the compositions.

In a further preferred embodiment, it may be advantageous to use mixtures of ascorbic acid and/or salts and/or derivatives of ascorbic acid with an extract of Olea europaea in the compositions according to the invention.

Mixtures of ascorbic acid, an ascorbic acid derivative known by the INCI name Ascorbyl Glucoside and a plant extract known by the INCI name Olea europaea Leaf Extract are more preferred within this embodiment. Such mixtures are commercially available, for example under the name Illumiscin® from Rahn. Illumiscin® may preferably be used in the compositions according to the invention as the skin-lightening active substance e).

In the event that the commercial product Illumiscin® is used as the skin-lightening active substance in the compositions according to the invention, the quantity thereof which is used is 0.10 to 8.0 wt. %, more preferably from 0.25 to 7.0 wt. %, more preferably from 0.50 to 6.0 wt. %, particularly preferably from 0.75 to 5.0 wt. % and in particular from 1.0 to 4.0 wt. %, in each case relative to the total weight of the compositions.

A further preferred skin-lightening active substance e), which can be excellently combined with the further components a) to d), is selected from 2-(4-hydroxyphenoxy)propionic acid and/or salts of 2-(4-hydroxyphenoxy)propionic acid. 2-(4-Hydroxyphenoxy)propionic acid and the salts thereof are commercially offered for sale in racemic form, as well as in the form of the R- and S-enantiomers thereof 2-(4-Hydroxyphenoxy)propionic acid and/or salts of 2-(4-hydroxyphenoxy)propionic acid may be used in the compositions according to the invention, preferably relative to the total weight thereof, preferably in quantities of 0.10 to 3.0 wt. %, more preferably of 0.20 to 2.50 wt. %, more preferably of 0.30 to 2.0 wt. %, particularly preferably of 0.40 to 1.50 wt. % and in particular of 0.50 to 1.25 wt. %.

According to the invention, 2-(4-hydroxyphenoxy)propionic acid is taken to mean not only the racemic mixture but also (S)-2-(4-hydroxyphenoxy)propionic acid or (R)-2-(4-hydroxyphenoxy)propionic acid.

(R)-2-(4-Hydroxyphenoxy)propionic acid or a physiologically acceptable salt such as alkali metal or ammonium salts of (R)-2-(4-hydroxyphenoxy)propionic acid is preferred with regard to its excellent efficacy against pigment spots of all kinds (age spots and/or freckles).

Corresponding products are commercially available, for example under the trade name Radianskin® PW LS 9918 from BASF.

It has been found that treating strongly pigmented skin with the compositions according to the invention led to particularly good results with regard to the lightening of pigment spots if ascorbic acid and/or the derivatives and/or salts thereof was/were combined with olive leaf extract and (R)-2-(4-hydroxyphenoxy)propionic acid. In particular, it consequently proved possible to achieve a considerable reduction in the lipofuscin content of the skin and thus also improved lightening or alleviation of age spots.

A combination of the commercial products Illumiscin® and Radianskin® PW LS 9918 was particularly suitable in this connection.

In a further preferred embodiment, cosmetic compositions are accordingly characterised in that they include
ascorbic acid and Ascorbyl Glucoside,
at least one plant extract known by the INCI name Olea europaea Leaf Extract, and
(R)-2-(4-hydroxyphenoxy)propionic acid and/or the alkali metal or ammonium salt thereof.

Resorcinol derivatives, in particular the compounds known by the INCI name Phenylethyl Resorcinol, and/or saturated or unsaturated, linear C₉₋₁₈ dicarboxylic acid (salts), may furthermore serve as skin-lightening active substances e) for the purposes of the present invention which may in each case be used in the compositions according to the invention preferably in quantities of 0.01 to 3.0 wt. %, more preferably of 0.05 to 2.50 wt. %, more preferably of 0.10 to 2.0 wt. %, particularly preferably of 0.20 to 1.50 wt. % and in particular of 0.30 to 1.0 wt. %, wherein the indications of quantity relate to the total weight of the compositions.

More suitable saturated or unsaturated, linear C₉₋₁₈ dicarboxylic acid (salts) may preferably be selected from: 8-hexadecene-1,16-dicarboxylic acid, 7-tetradecene-1,14-dicarboxylic acid, 9-octadecene-1,18-dicarboxylic acid, 6-dodecene-1,12-dicarboxylic acid, 5-decene-1,10-dicarboxylic acid, decanedioic acid (sebacic acid) and/or nonanedioic acid (azelaic acid).

8-Hexadecene-1,16-dicarboxylic acid (INCI name Octadecenedioic Acid) is a metabolic product of yeast cells from selected mutant strains of the Candida strain, the starting substance being a fatty acid of purely plant-based origin which is converted into the hydroxy fatty acid which is then oxidised via the fatty acid aldehyde stage to yield the dicarboxylic acid. The commercial product has a purity of 95%, the 8-hexadecene-1,16-dicarboxylic acid being present therein as a mixture of cis and trans isomers with the cis isomer predominating quantitatively. The product may include up to 3 wt. % oleic acid.

Particularly preferred compositions include, relative to the total weight thereof, 0.05 to 3.0 wt. %, preferably 0.07 to 2.0 wt. %, more preferably 0.08 to 1.50 wt. % and in particular 0.10 to 1.0 wt. % of 8-hexadecene-1,16-dicarboxylic acid (INCI name Octadecenedioic Acid).

In a further preferred embodiment, cosmetic compositions are characterised in that they include as skin-lightening active substance e) at least one compound known by the INCI name Phenylethyl Resorcinol and/or at least one linear C₉₋₁₈ dicarboxylic acid, preferably 8-hexadecene-1,16-dicarboxylic acid, 7-tetradecene-1,14-dicarboxylic acid, 9-octadecene-1,18-dicarboxylic acid, 6-dodecene-1,12-dicarboxylic acid, 5-decene-1,10-dicarboxylic acid, decanedioic acid (sebacic acid) and/or nonanedioic acid (azelaic acid), particularly preferably Phenylethyl Resorcinol and/or 9-octadecene-1,18-dicarboxylic acid (INCI name: Octadecenedioic Acid).

More preferred skin-lightening active substances e) are those from the group ascorbic acid, 2-(4-hydroxyphenoxy)propionic acid, Phenylethyl Resorcinol, Octadecenedioic Acid, Olea europaea Extract, Ascorbyl Tetraisopalmitate, Magnesium Ascorbyl Phosphate and Ascorbyl Glucoside.

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

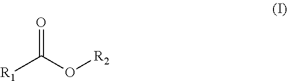

the residues R₁ and R₂ mutually independently denoting an unsaturated alkyl residue with 16 to 26 carbon atoms, and
e) 0.02 to 9.0 wt. % of at least one skin-lightening active substance selected from the group ascorbic acid, 2-(4-hydroxyphenoxy)propionic acid, Phenylethyl Resorcinol, Octadecenedioic Acid, Olea europaea Extract, Ascorbyl Tetraisopalmitate, Magnesium Ascorbyl Phosphate and Ascorbyl Glucoside.

Preferred compositions include at least one organic UV filter as a further cosmetic active substance.

Through the addition of a UV filter, both the agents themselves and the treated fibres may be protected from the harmful effects of UV radiation. At least one UV filter is accordingly preferably added to the agent. Suitable UV filters are not subject to any general restrictions with regard to structure and physical properties. Rather, any UV filters usable in the field of cosmetics whose absorption maximum is in the UVA (315-400 nm), the UVB (280-315 nm) or the UVC (<280 nm) range are suitable. UV filters with an absorption maximum in the UVB range, in particular in the range from approximately 280 to approximately 300 nm, are more preferred.

Examples of UV filters usable according to the invention are 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts thereof, (Phenylbenzimidazole Sulfonic Acid; Parsol®HS; Neo Heliopan® Hydro), 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)-propane-1,3-dione (Butyl Methoxydibenzoylmethane; Parsol®1789, Eusolex®9020), α-(2-oxoborn-3-ylidene)-toluene-4-sulfonic acid and the salts thereof, ethoxylated 4-aminobenzoic acid ethyl ester (PEG-25 PABA; Uvinul®P 25), 4-dimethylaminobenzoic acid 2-ethylhexyl ester (Octyl Dimethyl PABA; Uvasorb®DMO, Escalol®507, Eusolex®6007), salicylic acid 2-ethylhexyl ester (Octyl Salicylate; Escalol®587, Neo Heliopan®OS, Uvinul®O18), 4-methoxycinnamic acid isopentyl ester (Isoamyl p-Methoxycinnamate; Neo Heliopan®E 1000), 4-methoxycinnamic acid 2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene Camphor; Parsol®5000, Eusolex®6300), 3-benzylidenecamphor (3-Benzylidene Camphor), 4-isopropylbenzyl salicylate 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide, 2,4-dihydroxybenzophenone (Benzophenone-1; Uvasorb®20 H, Uvinul®400), 1,1′-diphenylacrylonitrile acid 2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzo-phenone-5-sodium sulfonate, 2-cyano-3,3-diphenylacrylic acid-2′-ethylhexyl ester and phenylene-1,4-bis-(2-benzimidazole)-3,3′-5,5′-tetrasulfonic acid-bis-sodium salt (Disodium Phenyl Dibenzimidazole Tetrasulfonate, Neo Heliopan® AP).

UV filters which are preferred according to the invention are substituted dibenzoylmethanes, diphenylacrylic acid esters, salicylic acid esters and benzimidazoles.

More preferred organic UV filters are
3,3,5-trimethylcyclohexyl salicylate,
2-phenylbenzimidazole-5-sulfonic acid and the salts thereof,
1-(4-tert.-butylphenyl)-3-(4-methoxyphenyl)propane-1,3-dione,
1,1′-diphenylacrylonitrile acid 2-ethylhexyl ester, preferably in combination with ethylenediaminetetraacetic acid and the salts thereof,
phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine.

It goes without saying that combinations of organic UV filters may also be used in the cosmetic compositions according to the invention. Preferred combinations of UV filters here in particular include the UV filters previously described as more preferred. Thanks to their excellent physical stability, the cosmetic compositions according to the invention are particularly well suited for formulating a plurality of organic UV filters.

It is more preferred for the organic UV filter to include at least two, preferably at least three and in particular at least four organic UV filters from the group 3,3,5-trimethylcyclohexyl salicylate, 2-phenylbenzimidazole-5-sulfonic acid and the salts thereof, 1-(4-tert.-butylphenyl)-3-(4-methoxyphenyl)propane-1,3-dione, 1,1′-diphenylacrylonitrile acid 2-ethylhexyl ester and phenylene-1,4-bis-(2-benzimidazole)-3,3′-5,5′-tetrasulfonic acid and the salts thereof.

The proportion by weight of the organic UV filters in the total weight of the compositions preferably amounts to from 1.0 to 35 wt. %, preferably from 2.0 to 30 wt. % and in particular from 5.0 to 25 wt. %.

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

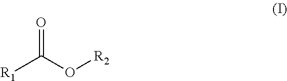

the residues R₁ and R₂ mutually independently denoting an unsaturated alkyl residue with 16 to 26 carbon atoms, and
e) 0.02 to 9.0 wt. % at least one skin-lightening active substance selected from the group ascorbic acid, 2-(4-hydroxyphenoxy)propionic acid, Phenylethyl Resorcinol, Octadecenedioic Acid, Olea europaea Extract, Ascorbyl Tetraisopalmitate, Magnesium Ascorbyl Phosphate and Ascorbyl Glucoside,
f) 1.0 to 35 wt. % of at least one organic UV filter from the group
   3,3,5-trimethylcyclohexyl salicylate,
   2-phenylbenzimidazole-5-sulfonic acid and the salts thereof,
   1-(4-tert.-butylphenyl)-3-(4-methoxyphenyl)propane-1,3-dione,
   1,1′-diphenylacrylonitrile acid 2-ethylhexyl ester, preferably in combination with ethylenediaminetetraacetic acid and the salts thereof,
   phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine.

The compositions according to the invention include a linear alkane with 11 to 18 carbon atom as a further preferred optional component. Preferred linear alkanes are those which include 11 to 13 carbon atoms, it being more preferred for the linear alkane to include 11 and/or 13 carbon atoms.

The use of mixtures of undecane and tridecane is more preferred. The use of mixtures of undecane and tridecane is more preferred, in which the two linear alkanes are present in a weight ratio of 4:1 to 1:1, in particular of 3:1 to 1:1. Corresponding mixtures of substances are for example obtainable under the trade name Cetiol Ultimate.

Not only does the addition of the linear alkanes improve the storage stability of the cosmetic compositions, but addition of the above-described alkanes also improves the solubility of oil-soluble UV filters, improves the spreadability of the composition on the skin, reduces the tackiness of the composition and improves skin feel once the composition has been applied to the skin.

Cosmetic compositions in which the linear alkane is present, relative to the total weight of the compositions, in quantities of 0.1 to 10 wt. %, preferably of 0.2 to 7.0 wt. % and in particular of 0.5 to 5.0 wt. % are preferred.

In order to establish a suitable viscosity and to further improve storage stability, preferred cosmetic compositions furthermore include
at least one hydrophobically modified (meth)acrylic acid copolymer and/or
at least one copolymer of 2-methyl-2[(1-oxo-2-propenyl)amino]-1-propanesulfonic acid. It is more preferred to use a combination of both of the above-stated polymers.

The hydrophobically modified (meth)acrylic acid copolymers used are preferably copolymers which are attributable to
at least one first monomer from the group of unsaturated carboxylic acids and unsaturated carboxylic acid esters, and
at least one second monomer from the group of unsaturated hydrophobically modified monomers.

Preferred copolymers are based on at least one first monomer from the group acrylic acid, methacrylic acid, C₁-C₆ alkyl acrylic acid esters and C₁-C₆ alkyl methacrylic acid esters. The acrylic acid esters and methacrylic acid esters are preferably esters of the respective acids with non-tertiary alkyl alcohols having alkyl residues with 1 to 12 carbon atoms, in particular 2 to 4 carbon atoms. Suitable monomers which may be mentioned by way of example are ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate.

The group of hydrophobically modified second monomers denotes monomers which have a hydrophobic substructure. Preferred monomers may in turn be attributable to the following two structural units:
an unsaturated acid, preferably acrylic acid, methacrylic acid or itaconic acid;
a C₈₋₄₀ alkyl chain, preferably a C₁₀₋₃₀ alkyl chain.

These two substructures may optionally be supplemented by a third structural unit from the group of polyoxyalkylene groups, preferably of polyethylene glycol groups, polypropylene glycol groups or polyethylene glycol/polypropylene glycol groups.

C₁₀₋₃₀ alkyl acrylates, C₁₀₋₃₀ alkyl PEG acrylates, C₁₀₋₃₀ alkyl PEG methacrylates or C₁₀₋₃₀ alkyl PEG itaconates are, for example, used as the second monomer. Preferred monomers are selected from C₁₀₋₃₀ alkyl acrylates, C₁₀₋₃₀ alkyl PEG 20-25 acrylates, C₁₀₋₃₀ alkyl PEG 20-25 methacrylates or C₁₀₋₃₀ alkyl PEG 20-25 itaconates. More preferred monomers are selected from the group of C₁₀₋₃₀ alkyl acrylates, Steareth-20 methacrylates, Beheneth-25 methacrylates, Steareth-20 itaconates, Ceteth-20 itaconates, Palmeth-25 acrylates or C₁₀₋₃₀ alkyl PEG-20 itaconates.

In summary, preferred hydrophobically modified (meth)acrylic acid copolymers are those which are attributable to
at least one monomer from the group acrylic acid, methacrylic acid, C₁-C₆ alkyl acrylic acid esters, C₁-C₆ alkyl methacrylic acid esters, and
at least one monomer from the group of C₁₀₋₃₀ alkyl acrylates, C₁₀₋₃₀ alkyl methacrylates, C₁₀₋₃₀ alkyl PEG-acrylates, C₁₀₋₃₀ alkyl PEG methacrylates or C₁₀₋₃₀ alkyl PEG itaconates, preferably from the group of C₁₀₋₃₀ alkyl acrylates.

More preferred hydrophobically modified (meth)acrylic acid copolymers are selected from the group of compounds with the INCI name Acrylates/C₁₀₋₃₀ Alkyl Acrylate Crosspolymer, as are for example obtainable under the trade names Carbopol ETD 2020, Ultrez 21 or Pemulen TR1.

The proportion by weight of the hydrophobically modified (meth)acrylic acid copolymer in the total weight of the composition preferably amounts to from 0.05 to 2.0 wt. %, preferably from 0.1 to 1.2 wt. % and in particular 0.2 to 0.8 wt. %.

Preferably used copolymers of 2-methyl-2[(1-oxo-2-propenyl)amino]-1-propanesulfonic acid are those copolymers which are attributable to
2-methyl-2[(1-oxo-2-propenyl)amino]-1-propanesulfonic acid and
vinylpyrrolidone.
   These copolymers are preferably crosslinked.

Crosslinked copolymers of vinylpyrrolidone and 2-methyl-2[(1-oxo-2-propenyl)-amino]-1-propanesulfonic acid are for example obtainable by copolymerising the two monomers in the presence of ammonium hydroxide and a crosslinking agent. Crosslinking may proceed with the assistance of olefinically polyunsaturated compounds, for example divinylbenzene, tetraallyloxyethane, methylenebisacrylamide, diallyl ether, polyallyl polyglyceryl ether or allyl ethers of sugars or sugar derivatives such as erythritol, pentaerythritol, arabitol, mannitol, sorbitol, sucrose or glucose. Methylenebisacrylamide is a preferred crosslinking agent.

One particularly suitable copolymer is the commercial product Aristoflex® AVC (from Clariant) which, as the ammonium salt, assumes the form of a white powder.

The copolymer of 2-methyl-2[(1-oxo-2-propenyl)amino]-1-propanesulfonic acid is preferably present in the compositions according to the invention in quantities of 0.05 to 3.0 wt. %, preferably of 0.1 to 2.0 wt. % and in particular of 0.2 to 1.5 wt. %.

### Summary Table

The following tables show the composition of some preferred cosmetic compositions (values stated in wt. % relative to the total weight of the cosmetic composition unless otherwise stated).

| | Formula 1 | Formula 2 | Formula 3 | Formula 4 |
|---|---|---|---|---|
| Monoglycerol fatty | 0.01 to 6.0 | 0.1 to 4.0 | 0.1 to 4.0 | 0.5 to 2.0 |
| acid ester A | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 5 | Formula 6 | Formula 7 | Formula 8 |
|---|---|---|---|---|
| Mono-, di- or triester | 0.01 to 6.0 | 0.1 to 4.0 | 0.1 to 4.0 | 0.5 to 2.0 |
| of glycerol with | | | | |
| behenic acid | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 11 | Formula 12 | Formula 13 | Formula 14 |
|---|---|---|---|---|
| Monoglycerol | 0.01 to 6.0 | 0.1 to 4.0 | 0.1 to 4.0 | 0.5 to 2.0 |
| fatty acid ester A | | | | |
| Di-, tri- or | 0.05 to 3.0 | 0.05 to 3.0 | 0.1 to 2.0 | 0.2 to 1.5 |
| tetraglycerol | | | | |
| difatty acid ester | | | | |
| of saturated or | | | | |
| unsaturated | | | | |
| C₁₆₋₁₈ fatty acids | | | | |
| Polyglycerol | 0.2 to 8.0 | 0.2 to 8.0 | 0.4 to 6.0 | 0.6 to 4.0 |
| difatty acid ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 15 | Formula 16 | Formula 17 | Formula 18 |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated or | | | | |
| unsaturated C₁₆₋₁₈ | | | | |
| fatty acids | | | | |
| Fatty acid ester | 0.05 to 3.0 | 0.1 to 2.0 | 0.1 to 2.0 | 0.2 to 1.5 |
| of the general | | | | |
| formula (I) | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 21 | Formula 22 | Formula 23 | Formula 24 |
|---|---|---|---|---|
| Monoglycerol | 0.01 to 6.0 | 0.1 to 4.0 | 0.1 to 4.0 | 0.5 to 2.0 |
| fatty acid ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Polyglycerol | 0.2 to 8.0 | 0.2 to 8.0 | 0.4 to 6.0 | 0.6 to 4.0 |
| difatty acid | | | | |
| ester C | | | | |
| Fatty acid ester | 0.05 to 3.0 | 0.1 to 2.0 | 0.1 to 2.0 | 0.2 to 1.5 |
| of the general | | | | |
| formula (I), | | | | |
| in which the | | | | |
| residues R₁ and | | | | |
| R₂ mutually | | | | |
| independently | | | | |
| denote an | | | | |
| unsaturated | | | | |
| C₁₆₋₂₆ alkyl | | | | |
| residue | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 25 | Formula 26 | Formula 27 | Formula 28 |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Polyglycerol | 0.2 to 8.0 | 0.2 to 8.0 | 0.4 to 6.0 | 0.6 to 4.0 |
| difatty acid | | | | |
| ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Skin-lightening | 0.02 to 9.0 | 0.03 to 8.0 | 0.04 to 7.0 | 0.05 to 6.0 |
| active substance¹⁾ | | | | |
| Water | 30 to 82 | 30 to 82 | 35 to 75 | 45 to 60 |
| Misc. | ad 100 | ad 100 | ad 100 | ad 100 |

| | Formula 31 | Formula 32 | Formula 33 | Formula 34 |
|---|---|---|---|---|
| Mono-, di- or | 0.01 to 6.0 | 0.1 to 4.0 | 0.1 to 4.0 | 0.5 to 2.0 |
| triester of glycerol | | | | |
| with behenic acid | | | | |
| Di-, tri- or | 0.05 to 3.0 | 0.05 to 3.0 | 0.1 to 2.0 | 0.2 to 1.5 |
| tetraglycerol | | | | |
| difatty acid ester | | | | |
| of saturated | | | | |
| or unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated or | | | | |
| unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), | | | | |
| in which the | | | | |
| residues R₁ and | | | | |
| R₂ mutually | | | | |
| independently | | | | |
| denote an | | | | |
| unsaturated | | | | |
| C₁₆₋₂₆ alkyl residue | | | | |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 35 | Formula 36 | Formula 37 | Formula 38 |
|---|---|---|---|---|
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Di-, tri- or | 0.05 to 3.0 | 0.05 to 3.0 | 0.1 to 2.0 | 0.2 to 1.5 |
| tetraglycerol | | | | |
| difatty acid ester | | | | |
| of saturated or | | | | |
| unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid | | | | |
| ester of saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), | | | | |
| in which the | | | | |
| residues R₁ | | | | |
| and R₂ mutually | | | | |
| independently | | | | |
| denote an | | | | |
| unsaturated | | | | |
| C₁₆₋₂₆ alkyl | | | | |
| residue | | | | |
| Skin-lightening | 0.02 to 9.0 | 0.03 to 8.0 | 0.04 to 7.0 | 0.05 to 6.0 |
| active substance¹⁾ | | | | |
| Water | 30 to 82 | 30 to 82 | 35 to 75 | 45 to 60 |
| Misc. | ad 100 | ad 100 | ad 100 | ad 100 |

| | Formula 41 | Formula 42 | Formula 43 | Formula 44 |
|---|---|---|---|---|
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Skin-lightening | 0.02 to 9.0 | 0.03 to 8.0 | 0.04 to 7.0 | 0.05 to 6.0 |
| active substance | | | | |
| Water | 30 to 82 | 30 to 82 | 35 to 75 | 45 to 60 |
| Misc. | ad 100 | ad 100 | ad 100 | ad 100 |

| | Formula 45 | Formula 46 | Formula 47 | Formula 48 |
|---|---|---|---|---|
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Skin-lightening | 0.02 to 9.0 | 0.03 to 8.0 | 0.04 to 7.0 | 0.05 to 6.0 |
| active substance¹⁾ | | | | |
| Water | 30 to 82 | 30 to 82 | 35 to 75 | 45 to 60 |
| Misc. | ad 100 | ad 100 | ad 100 | ad 100 |

| | Formula 1a | Formula 2a | Formula 3a | Formula 4a |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 5a | Formula 6a | Formula 7a | Formula 8a |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 11a | Formula 12a | Formula 13a | Formula 14a |
|---|---|---|---|---|
| Monoglycerol fatty acid ester A | 0.01 to 6.0 | 0.1 to 4.0 | 0.1 to 4.0 | 0.5 to 2.0 |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 15a | Formula 16a | Formula 17a | Formula 18a |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 21a | Formula 22a | Formula 23a | Formula 24a |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 25a | Formula 26a | Formula 27a | Formula 28a |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance¹⁾ | | | | |
| Water | 30 to 82 | 30 to 82 | 35 to 75 | 45 to 60 |
| Misc. | ad 100 | ad 100 | ad 100 | ad 100 |

| | Formula 31a | Formula 32a | Formula 33a | Formula 34a |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| Formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 35a | Formula 36a | Formula 37a | Formula 38a |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Skin-lightening active | 0.02 to 9.0 | 0.03 to 8.0 | 0.04 to 7.0 | 0.05 to 6.0 |
| substance¹⁾ | | | | |
| Water | 30 to 82 | 30 to 82 | 35 to 75 | 45 to 60 |
| Misc. | ad 100 | ad 100 | ad 100 | ad 100 |

| | Formula 41a | Formula 42a | Formula 43a | Formula 44a |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Skin-lightening | 0.02 to 9.0 | 0.03 to 8.0 | 0.04 to 7.0 | 0.05 to 6.0 |
| active | | | | |
| substance | | | | |
| Water | 30 to 82 | 30 to 82 | 35 to 75 | 45 to 60 |
| Misc. | ad 100 | ad 100 | ad 100 | ad 100 |

| | Formula 45a | Formula 46a | Formula 47a | Formula 48a |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Skin-lightening | 0.02 to 9.0 | 0.03 to 8.0 | 0.04 to 7.0 | 0.05 to 6.0 |
| active | | | | |
| substance¹⁾ | | | | |
| Water | 30 to 82 | 30 to 82 | 35 to 75 | 45 to 60 |
| Misc. | ad 100 | ad 100 | ad 100 | ad 100 |

| | Formula 1c | Formula 2c | Formula 3c | Formula 4c |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| Formula (I) | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 5c | Formula 6c | Formula 7c | Formula 8c |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 11c | Formula 12c | Formula 13c | Formula 14c |
|---|---|---|---|---|
| Monoglycerol fatty acid ester A | 0.01 to 6.0 | 0.1 to 4.0 | 0.1 to 4.0 | 0.5 to 2.0 |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| Formula (I) | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 15c | Formula 16c | Formula 17c | Formula 18c |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I) | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula 21c | Formula 22c | Formula 23c | Formula 24c |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 25c | 26c | 27c | 28c |
| Monoglycerol fatty acid | 0.01 to 6.0 | 0.1 to 4.0 | 0.1 to 4.0 | 0.5 to 2.0 |
| ester A | | | | |
| Polyglycerol difatty acid | 0.05 to 3.0 | 0.05 to 3.0 | 0.1 to 2.0 | 0.2 to 1.5 |
| ester B | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹) | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 31c | 32c | 33c | 34c |
| Mono-, di- or triester | 0.01 to 6.0 | 0.1 to 4.0 | 0.1 to 4.0 | 0.5 to 2.0 |
| of glycerol with | | | | |
| behenic acid | | | | |
| Di-, tri- or tetraglycerol | 0.05 to 3.0 | 0.05 to 3.0 | 0.1 to 2.0 | 0.2 to 1.5 |
| difatty acid ester of | | | | |
| saturated or unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol difatty | | | | |
| acid ester of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I), in | | | | |
| which the residues R₁ | | | | |
| and R₂ mutually | | | | |
| independently denote an | | | | |
| unsaturated C₁₆₋₂₆ | | | | |
| alkyl residue | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 35c | 36c | 37c | 38c |
| Mono-, di- or triester | 0.01 to 6.0 | 0.1 to 4.0 | 0.1 to 4.0 | 0.5 to 2.0 |
| of glycerol with | | | | |
| behenic acid | | | | |
| Di-, tri- or tetraglycerol | 0.05 to 3.0 | 0.05 to 3.0 | 0.1 to 2.0 | 0.2 to 1.5 |
| difatty acid ester of | | | | |
| saturated or unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol difatty | | | | |
| acid ester of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I), in | | | | |
| which the residues R₁ | | | | |
| and R₂ mutually | | | | |
| independently denote an | | | | |
| unsaturated C₁₆₋₂₆ | | | | |
| alkyl residue | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹) | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 41c | 42c | 43c | 44c |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Undecane, tridecane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 45c | 46c | 47c | 48c |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Undecane, tridecane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹) | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 1d | 2d | 3d | 4d |
| Monoglycerol fatty acid | 0.01 to 6.0 | 0.1 to 4.0 | 0.1 to 4.0 | 0.5 to 2.0 |
| ester A | | | | |
| Polyglycerol difatty acid | 0.05 to 3.0 | 0.05 to 3.0 | 0.1 to 2.0 | 0.2 to 1.5 |
| ester B | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 5d | 6d | 7d | 8d |
| Mono-, di- or triester | 0.01 to 6.0 | 0.1 to 4.0 | 0.1 to 4.0 | 0.5 to 2.0 |
| of glycerol with | | | | |
| behenic acid | | | | |
| Polyglycerol difatty acid | 0.05 to 3.0 | 0.05 to 3.0 | 0.1 to 2.0 | 0.2 to 1.5 |
| ester B | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 11d | 12d | 13d | 14d |
| Monoglycerol fatty acid | 0.01 to 6.0 | 0.1 to 4.0 | 0.1 to 4.0 | 0.5 to 2.0 |
| ester A | | | | |
| Di-, tri- or tetraglycerol | 0.05 to 3.0 | 0.05 to 3.0 | 0.1 to 2.0 | 0.2 to 1.5 |
| difatty acid ester of | | | | |
| saturated or unsaturated | | | | |
| C₁₆₋₁₈ fatty acids | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 15d | 16d | 17d | 18d |
| Monoglycerol fatty acid | 0.01 to 6.0 | 0.1 to 4.0 | 0.1 to 4.0 | 0.5 to 2.0 |
| ester A | | | | |
| Polyglycerol difatty acid | 0.05 to 3.0 | 0.05 to 3.0 | 0.1 to 2.0 | 0.2 to 1.5 |
| ester B | | | | |
| Penta-, hexa- or | 0.2 to 8.0 | 0.2 to 8.0 | 0.4 to 6.0 | 0.6 to 4.0 |
| heptaglycerol difatty | | | | |
| acid ester of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 21d | 22d | 23d | 24d |
| Monoglycerol fatty acid | 0.01 to 6.0 | 0.1 to 4.0 | 0.1 to 4.0 | 0.5 to 2.0 |
| ester A | | | | |
| Polyglycerol difatty acid | 0.05 to 3.0 | 0.05 to 3.0 | 0.1 to 2.0 | 0.2 to 1.5 |
| ester B | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I), in | | | | |
| which the residues R₁ | | | | |
| and R₂ mutually | | | | |
| independently denote an | | | | |
| unsaturated C₁₆₋₂₆ | | | | |
| alkyl residue | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 25d | 26d | 27d | 28d |
| Monoglycerol fatty acid | 0.01 to 6.0 | 0.1 to 4.0 | 0.1 to 4.0 | 0.5 to 2.0 |
| ester A | | | | |
| Polyglycerol difatty acid | 0.05 to 3.0 | 0.05 to 3.0 | 0.1 to 2.0 | 0.2 to 1.5 |
| ester B | | | | |
| Polyglycerol difatty acid | 0.2 to 8.0 | 0.2 to 8.0 | 0.4 to 6.0 | 0.6 to 4.0 |
| ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹) | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 31d | 32d | 33d | 34d |
| Mono-, di- or triester | 0.01 to 6.0 | 0.1 to 4.0 | 0.1 to 4.0 | 0.5 to 2.0 |
| of glycerol with | | | | |
| behenic acid | | | | |
| Di-, tri- or tetraglycerol | 0.05 to 3.0 | 0.05 to 3.0 | 0.1 to 2.0 | 0.2 to 1.5 |
| difatty acid ester of | | | | |
| saturated or unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol difatty | | | | |
| acid ester of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I), in | | | | |
| which the residues R₁ | | | | |
| and R₂ mutually | | | | |
| independently denote an | | | | |
| unsaturated C₁₆₋₂₆ | | | | |
| alkyl residue | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 35d | 36d | 37d | 38d |
| Mono-, di- or triester | 0.01 to 6.0 | 0.1 to 4.0 | 0.1 to 4.0 | 0.5 to 2.0 |
| of glycerol with | | | | |
| behenic acid | | | | |
| Di-, tri- or tetraglycerol | 0.05 to 3.0 | 0.05 to 3.0 | 0.1 to 2.0 | 0.2 to 1.5 |
| difatty acid ester of | | | | |
| saturated or unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol difatty | | | | |
| acid ester of saturated | | | | |
| or unsaturated C₁₆₋₁₈ | | | | |
| fatty acids | | | | |
| Fatty acid ester of the | 0.05 to 3.0 | 0.1 to 2.0 | 0.1 to 2.0 | 0.2 to 1.5 |
| general formula (I), in | | | | |
| which the residues R₁ | | | | |
| and R₂ mutually | | | | |
| independently denote an | | | | |
| unsaturated C₁₆₋₂₆ | | | | |
| alkyl residue | | | | |
| Linear C₁₄₋₁₈ fatty | 0.01 to 5.0 | 0.05 to 3.0 | 0.05 to 3.0 | 0.1 to 1.0 |
| alcohol | | | | |
| Linear C₁₁₋₁₃ alkane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹⁾ | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 41d | 42d | 43d | 44d |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Undecane, tridecane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 45d | 46d | 47d | 48d |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Undecane, tridecane | 0.1 to 10 | 0.2 to 7.0 | 0.2 to 7.0 | 0.5 to 5.0 |
| Skin-lightening active | 0.02 to 9.0 | 0.03 to 8.0 | 0.04 to 7.0 | 0.05 to 6.0 |
| substance¹⁾ | | | | |
| Water | 30 to 82 | 30 to 82 | 35 to 75 | 45 to 60 |
| Misc. | ad 100 | ad 100 | ad 100 | ad 100 |

¹⁾The Skin-lightening active substance is selected from the group of ascorbic acid, 2-(4-hydroxyphenoxy)propionic acid, Phenylethyl Resorcinol, Octadecenedioic Acid, Olea europaea Extract, Ascorbyl Tetraisopalmitate, Magnesium Ascorbyl Phosphate and Ascorbyl Glucoside.

As explained above, a combination of components a), b), c) and d) is particularly suitable for improving both the physical and the chemical stability of cosmetic UV screening agents.

The present application also provides the use of a combination including
a) at least one monoglycerol fatty acid ester A,
b) at least one first polyglycerol difatty acid ester B,
c) at least one second polyglycerol difatty acid ester C which differs from the first polyglycerol difatty acid ester,
d) at least one fatty acid ester of the general formula (I)

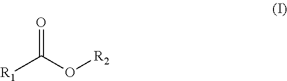

the residues R₁ and R₂ mutually independently denoting a saturated or unsaturated alkyl residue with 16 to 26 carbon atoms,
for
   improving the storage stability of a cosmetic composition including at least one skin-lightening active substance;
   improving the UV-protective action of a cosmetic composition including at least one skin-lightening active substance;
   improving the absorbability of a cosmetic composition including at least one skin-lightening active substance;
   improving the spreadability and reducing the tackiness of a cosmetic composition including at least one skin-lightening active substance.

The present invention thirdly and finally provides a method for cosmetic skin care, in which a cosmetic composition according to the invention is applied onto the skin, in particular the facial skin.

The statements made regarding the cosmetic agents according to the invention apply mutatis mutandis to the use according to the invention and to the cosmetic method according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition in the form of an oil-in-water emulsion including:
a) at least one monoglycerol fatty acid ester A,
b) at least one first polyglycerol difatty acid ester B,
c) at least one second polyglycerol difatty acid ester C which differs from the first polyglycerol difatty acid ester B,
d) at least one fatty acid ester of the general formula (I) 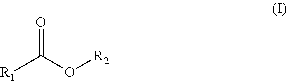
the residues R₁ and R₂ mutually independently denoting a saturated or unsaturated alkyl residue with 16 to 26 carbon atoms, and
e) at least one skin-lightening active substance.

2. The cosmetic composition according to claim 1, wherein the monoglycerol fatty acid ester A is present, relative to the total weight of the compositions, in quantities of 0.01 to 6.0 wt. %.

3. The cosmetic composition according to claim 1, wherein the first polyglycerol difatty acid ester B is present, relative to the total weight of the compositions, in quantities of 0.05 to 3.0 wt. %.

4. The cosmetic composition according to claim 1, wherein the second polyglycerol difatty acid ester C is present, relative to the total weight of the compositions, in quantities of 0.2 to 8.0 wt. %.

5. The cosmetic composition according to claim 1, wherein the fatty acid ester of the general formula (I) is present, relative to the total weight of the compositions, in quantities of 0.05 to 3.0 wt. %.

6. The cosmetic composition according to claim 1, wherein the skin-lightening active substance e) is present, relative to the total weight of the compositions, in quantities of 0.02 to 9.0 wt. %.

7. The cosmetic composition according to claim 1, further including
f) a linear fatty alcohol with 14 to 18 carbon atoms.

8. The cosmetic composition according to claim 1, further including
g) a linear alkane with 11 to 18 carbon atoms.

9. A method for cosmetic skin care, including applying onto the skin the cosmetic composition according to claim 1.
